(19) United States
(12) Patent Application Publication
(10) Pub. No.: US 2006/0275224 A1
(43) Pub. Date: Dec. 7, 2006

(54) Calcium carbonate compositions for use in the manufacture of toothpaste

(76) Inventors: James Gavin Burnet, Bebington (GB); Mark Flanagan, Bebington (GB); Donald Peter Gregory, Bebington (GB); Christopher Leonard Mackie, Montrose (GB); Jeffrey Price, Bebington (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/551,823

(22) PCT Filed: Mar. 16, 2004
(86) PCT No.: PCT/EP04/02849

§ 371 Date: Jul. 10, 2006

(30) Foreign Application Priority Data
Apr. 4, 2003 (EP) 03252171.8

(51) Int. Cl. A61K 8/21

(52) U.S. Cl. 424/52

(57) ABSTRACT

Process for the manufacture of an oral composition, said oral composition comprising from 5 to 60% by weight calcium carbonate as abrasive, said method characterised by the preparation of a slurry which comprises substantially all of the ingredients present in said oral composition followed by the addition of a thickening mixture to form said oral composition.

The present invention relates to a process for the manufacture of a calcium carbonate containing oral composition.

WO 00/69401 (Colgate) discloses a method of manufacturing a toothpaste composition, said method involving the formation of a calcium carbonate slurry to which is added a thickening mixture and thereafter any further ingredients to produce the toothpaste. There is no disclosure that the thickening mixture is the last item to be added in the manufacture to the toothpaste.

WO 02/13774 (Qimica Industrial Barra do Pirai) discloses an aqueous suspension of particulate calcium carbonate for use in the manufacture of a toothpaste composition, wherein the biological contamination of the suspension is not greater than the maximum allowable levels indicated by the US Food and Drugs Administration (FDA). There is no disclosure that the thickening mixture is the last item to be added in the manufacture to the toothpaste.

The present invention aims to provide an improved process for the manufacture of an oral composition which comprises as abrasive calcium carbonate. The use of low viscosity fluids in the manufacture of such oral compositions allows for longer storage periods of said fluids and also better processing management. In contrast to toothpaste compositions, slurries can be stored for longer periods at room temperature and pressure whereas toothpastes generally become gradually thicker after around 5 to 7 days. Slurries are also more easily transported to, from and around processing machinery with any air being trapped in the slurry being easily removed by natural handling and without intervention. Toothpastes on the other hand need physical intervention to remove air trapped within during handling. The present invention allows the manufacture of a near complete toothpaste composition which can be stored and processed without difficulty until transformation into the final oral composition is required.

Accordingly, the present invention provides a process for the manufacture of an oral composition, said oral composition comprising from 5 to 60% by weight calcium carbonate as abrasive, said method characterised by the preparation of a slurry which comprises substantially all of the ingredients present in said oral composition followed by the addition of a thickening mixture to form said oral composition.

In essence the process involves two steps. First, the manufacture of a slurry, and second, the thickening of the slurry to form the oral composition.

Accordingly, the slurry should, immediately before addition of the thickening mixture, comprise substantially all the materials present in the oral composition.

The thickening mixture comprises ingredients suitable to transform the slurry into an oral composition, preferably a toothpaste. No further ingredients should be necessary to impart a technical effect on the oral composition. However, ingredients which do not necessarily have an intrinsic thickening effect may be added either before or together with the thickening mixture. For example, surfactant and fluoride ion sources may be added to the slurry before or together with the thickening mixture. Typically, aqueous solutions will be added separately from the thickening mixture whereas solid additives will preferably be added within the thickening mixture. Surfactants such as SLS may be added as aqueous solutions or as solids so there is a degree of flexibility with the process. Further they may be mixed in with the thickening mixture so that all thickening mixture ingredients are added at the same time. Where surfactant or fluoride ion sources are added to the slurry before or together with the thickening mixture they should preferably be added as an aqueous solution. Where SLS is the surfactant the maximum aqueous concentration is 45%, preferably 35% and for fluoride ion source the maximum concentration is 42%.

As discussed above the thickening mixture may comprise materials which have no intrinsic thickening effect of their own so it is essential that some materials included in the thickening mixture do in fact have a thickening effect on addition to the slurry.

Thickeners which can be used in accordance with the process of the present invention preferably include the natural and synthetic gums and gum-like materials, desirably carboxyl methyl cellulose sodium carboxymethylcellulose, hydroxyethyl carboxymethylcellulose, carrageenan, gum tragacanth, xanthan gum, guar gum, alginates, bentonite and other natural clays and synthetic inorganic clays. The gums are hydratable or gelled with water or alkanols, especially with polyhydric alcohols such as glycerol and sorbitol.

The proportions of the thickening mixture present in the final oral composition of the present invention will generally be in the range from 0.1 to 5% by weight of the final oral care product and in the case of synthetic gums such as sodium carboxymethylcellulose, the range will preferably be from 0.1 to 3%.

Typical of the materials added to the slurry within the thickening mixture include any water-insoluble materials required in the oral composition. For example, flavours, antibacterials such as Triclosan and colours, etc.

Particularly preferred sodium carboxymethyl celluloses (SCMCs) include those with a degree of substitution of from 0.6 to 0.99, preferably from 0.7 to 0.95.

Further, preferred SCMCs include those with a viscosity of from 250 mPa·s to 10 000 mPa·s as measured on a Brookfield viscometer TA spindle at 30 rpm, 23° C. and reading after 30 seconds when slurried with flavour in a 1:1 ratio.

The calcium carbonate slurry prepared in accordance with the present invention is an aqueous suspension of calcium carbonate. The slurry will generally be present in the final product at from 5 to 65% by weight. Humectants are preferably present in the slurry. Suitable humectants used in dentifrice formulations are well known in the art and include glycerine, sorbitol, propylene glycol, polyethylene glycol, mannitol, polypropylene glycols, and mixtures thereof. A particularly preferred humectant is sorbitol and is preferably present at from 5 to 35% by weight of the final oral composition.

Stable aqueous calcium carbonate slurries used in the method of the present invention generally contain about 50 to about 80% by weight calcium carbonate. Such slurry materials are available commercially and are widely used in the paper making industry; calcium carbonate being a pigment which is excellent in whiteness and has affinity for ink, gloss and printability.

The slurry may also comprise any further ingredients used in oral care compositions including:

antimicrobial agents, e.g. Triclosan, chlorhexidine, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

desensitising agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

biomolecules, e.g. bacteriocins, antibodies, enzymes, etc.;

flavours, e.g. peppermint and spearmint oils;

proteinaceous materials such as collagen;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition.

humectants such as glycerol, sorbitol, propyleneglycol, xylitol, lactitol etc.;

binders and thickening mixtures such as sodium carboxymethyl-cellulose, xanthan gum, gum arabic etc. as well as synthetic polymers such as polyacrylates and carboxyvinyl polymers such as Carbopol®;

polymeric compounds which can enhance the delivery of active ingredients such as antimicrobial agents can also be included;

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

Accordingly, the slurry contains substantially all the materials intended to be present in the final oral composition, save those intended to be incorporated in or along with the thickening mixture.

The calcium carbonate used in the present invention may be natural calcium carbonate ground to form a powder or precipitated calcium carbonate. Such materials are well known in the art.

In a preferred embodiment the slurry contains a dispersant. Preferable dispersants include organic surfactants and suitable organic surfactants include the alkali-metal alkyl sulphates, e.g. sodium lauryl sulphate (SLS). Such a surfactant would be preferably present in the slurry at from 0.05 to 3% by weight of the slurry. The dispersant may also be inorganic dispersants which can be used to stabilise the calcium carbonate slurry. Suitable inorganic dispersants include such condensed phosphates as pyrophosphates, tripolyphosphates, trimetaphosphates, tetrametaphosphates, and hexametaphosphates, zinc salts and silicates. Organic dispersants, include polycarboxylates such as polyacrylates, polymethacrylates, and polymaleates and polyvinyl alcohol. Such dispersants are known to the art, for example, U.S. Pat. No. 4,818,783 discloses dispersing calcium carbonate in an aqueous medium containing as the dispersant (1) 0.1 to 2 parts by weight of (a) a carboxyl group-containing water-soluble polymer possessing a number average molecular weight in the range of 2,000 to 80,000 and (b) a water soluble condensed phosphate and (2) 0.03 to 1 part by weight of a water soluble anionic modified polyvinyl alcohol respectively based on 100 parts by weight of the calcium carbonate.

In a preferred embodiment the slurry comprises from 0.05 to 0.15% by weight SLS while the thickening mixture comprises enough SLS to produce a final SLS content of from 1.5 to 3.0% by weight of the final oral care composition.

Should a fluoride ion source such as sodium monofluorophosphate (SMFP) be included in the slurry it is preferable that it is added only after a suitable calcium ion chelator such as trisodium phosphate. This prevents the SMFP bonding with the calcium ions in the slurry.

The oral compositions may be in any form common in the art, e.g. toothpaste, gel, mousse, aerosol, gum, lozenge, cream, etc. and may also be formulated into systems for use in dual-compartment type dispensers. Preferably the oral composition is a toothpaste.

The process according to the invention involves the preparation of a slurry and also the formation of the final oral composition by the addition of the thickening mixture. The preparation of the slurry preferably involves the addition of the calcium carbonate to a water/humectant mixture. To this suspension of calcium carbonate in water/humectant can be added the remaining ingredients to be found in the oral composition save those present in the thickening mixture.

The final slurry can be stored in a mixing vessel at above 15° C. until it is needed. This preparation of the slurry may be done under vacuum or at atmospheric pressure. If the slurry is not prepared under a vacuum it is preferable that water-soluble materials such as SMFP an SLS are added as aqueous solutions. In a most preferred embodiment aqueous SLS is the last ingredient added before the thickening mixture.

The addition of the thickening mixture is the last step in the manufacture of the oral composition. The addition of the thickening mixture is usually although not necessarily done in a vacuum, e.g. by way of a coaxial injector. Adding the thickening mixture under a vacuum ensures de-aeration to the final product. Typically the subsequent mixing under a vacuum also de-aerates the final product.

In a further aspect the invention provides a continuous process for the manufacture of an oral composition said process comprising the following steps:
(a) preparation of a slurry as previously described;
(b) optional addition of further ingredients to said slurry;
(c) optional de-aeration of said slurry;
(d) addition of thickening mixture;
(e) mixing of slurry and thickening mixture;
(f) standing for from 30 seconds to 10 minutes or for long enough for the thickening mixture to hydrate;
(g) mixing for from 30 seconds to 10 minutes.

Preferably the thickening mixture is added under vacuum by way of a coaxial injector.

Preferably period (f) lasts for from 45 seconds to 6 minutes, more preferably from 1 minute to 4 minutes. Preferably period (g) lasts for from 45 seconds to 6 minutes, more preferably from 1 minute to 4 minutes.

1. Process for the manufacture of an oral composition, said oral composition comprising from 5 to 60% by weight calcium carbonate as abrasive, said method characterised by the preparation of a slurry which comprises substantially all of the ingredients present in said oral composition followed by the addition of a thickening mixture to form said oral composition.

2. Process according to claim 1, wherein the thickening mixture comprises a polymeric thickening mixture selected from carboxyl methyl cellulose, sodium carboxymethylcellulose, hydroxyethyl carboxymethylcellulose, carrageenin, gum tragacanth, xanthan gum, guar gum, alginates, bentonite and other natural clays and synthetic inorganic clays.

3. Process according to claim 1, wherein the thickening mixture comprises flavour at from 0.001 to 100% by weight of the final composition.

4. Process according to claim 1, wherein the thickening mixture comprises surfactant.

5. Process according to claim 1, wherein the thickening mixture comprises a fluoride ion source.

6. Process according to claim 1, wherein the thickening mixture comprises from 0.1 to 20% by weight of the oral composition.

7. Process according to claim 1, wherein the slurry comprises an aqueous suspension of calcium carbonate.

8. Process according to claim 1, wherein the slurry comprises a humectant.

9. Process according to claim 1, wherein the slurry comprises a thickening silica.

10. Process according to claim 1, wherein the slurry comprises dispersant.

11. Process according to claim 1, wherein the slurry comprises a fluoride ion source.

12. Process according to claim 1, wherein the slurry is prepared by adding calcium carbonate to a water/humectant mixture.

13. A continuous process for the manufacture of an oral composition, said method characterised by the following steps:
(a) preparation of an aqueous suspension of calcium carbonate;
(b) optional addition of further ingredients to said suspension;
(c) optional de-aeration of said suspension;
(d) addition of thickening mixture;
(e) mixing of said suspension and thickening mixture;
(f) standing for from 30 seconds to 10 minutes or for long enough for the thickening mixture to hydrate;
(g) mixing for from 30 seconds to 10 minutes.
